(19) United States
(12) Patent Application Publication
(10) Pub. No.: US 2004/0111139 A1
(43) Pub. Date: Jun. 10, 2004

(54) Apparatus and methods for differential stimulation of nerve fibers

(76) Inventor: Douglas B. McCreery, Pasadena, CA (US)

Correspondence Address: Michael J. Farber, Esq., PAUL, HASTINGS, JANOFSKY & WALKER, LLP, 12390 El Camino Real, San Diego, CA, 92130, US

(21) Appl. No.: 10/315,421

(22) Filed: Dec. 10, 2002

(51) Int. Cl. A61N 1/05

(52) U.S. Cl. 607/117

(57) ABSTRACT

An implantable helical electrode assembly configured to fit around a nerve for electrically triggering or measuring an action potential or for blocking conduction in nerve tissue. A multiconductor flexible cable connects the electrode to an implanted signal receiver, and the assembly may include multiple individual flexible ribbon electrodes each partially embedded in a portion of the peripheral surface of a helically formed dielectric support matrix. The helical electrode assembly has a helix of a selected pitch and with a selected number of helical turns so that the entire peripheral nerve or any portion of it extending radially inward from the surface forming an annulus can be electrically stimulated. The spiral configuration of the assembly is easy to install around a nerve bundle during surgical implantation, and the resiliency of the assembly minimizes the risk of damage to nerve tissue. The tissue-contacting surface of each electrode is roughened to increase the electrode surface area.

### BACKGROUND OF THE INVENTION

It has been known for almost 200 years that muscle contraction can be controlled by applying an electrical stimulus to the associated nerves. Practical long-term application of this knowledge, however, was not possible until the relatively recent development of totally implantable miniature electronic circuits which avoid the risk of infection at the sites of percutaneous connecting wires. A well-known example of this modern technology is the artificial cardiac pacemaker that has been successfully implanted in many patients.

Modern circuitry enables wireless control of implanted devices by wireless telemetry communication between external and internal circuits. That is, external controls can be used to command implanted nerve stimulators to regain muscle control in injured limbs, to control bladder and sphincter function, to alleviate pain and hypertension, and to restore proper function to many other portions of an impaired or injured nerve-muscle system.

To provide an electrical connection to the peripheral nerve which controls the muscles of interest, an electrode (and sometimes an array of multiple electrodes) is secured to and around the nerve bundle. A wire or cable from the electrode is in turn connected to the implanted package of circuitry. The present invention is directed to an improvement in this type of an electrode.

A widely used prior-art electrode assembly is formed from a tube of silicon rubber with one or more electrodes secured on the inner surface of the tube. An end-to-end slit is cut through the tube sidewall so that the tube can be opened and fitted over the nerve bundle. When so installed, the resiliency of the tube causes it to surround the nerve bundle to urge the electrode against the surface of the tissue. The tube may also be provided with suture flaps for additional anchorage. Due to its construction, this style of assembly is usually called a “cuff” electrode. Animal-implant studies suggest that cuff electrodes can cause nerve damage, and are not wholly satisfactory for long-term implantation. The probable causes of these problems can be summarized as follows:

(1) Although having some radial flexibility to enable installation over the nerve, the silicon-rubber tube or sleeve is relatively stiff to ensure that the restoring force of the resilient material will position the electrode against the nerve surface to ensure adequate electrical contact. Excessive gripping and compression of the nerve by the cuff can cause nerve damage by decreasing blood and axoplasmic flow, and by constricting nerve fibers with resulting loss of function. This problem is accentuated by temporary swelling of the nerve caused by the trauma of surgical implantation of the electrode.

(2) If a cuff electrode is loosely fitted to limit pressure atrophy of the nerve, a poor electrical contact is made, and this contact is further degraded in time by ingrowth of connective tissue between the cuff and nerve. This ingrowth is sometimes sufficiently marked to lead to compression damage to the nerve, as discussed above, or it may cause complete separation of the cuff and nerve.

(3) The nerve is encased within the full length of the cuff, blocking a normal metabolic exchange between the nerve and surrounding tissue. That is, a normal and desired fluid interchange between the nerve and its surrounding environment is prevented or sharply decreased over the length of the cuff.

(4) In addition to compression damage, mechanical trauma to the nerve can be caused by torque or bending forces applied to the cuff and its relatively stiff cable during muscle and body movement. These forces may even displace the nerve bundle out of the cuff.

(5) Conventional cuff assemblies use electrodes of small surface area, and the resulting high density of electrical charge at the electrode-nerve interface can result in an undesired electrochemical deposition of electrode material on the nerve sheath.

A number of solutions have been proposed, including electrodes disclosed in the following United States patents: U.S. Pat. No. 3,760,812 to Timm et al.; U.S. Pat. No. 4,026,303 to Babotai; U.S. Pat. No. 4,979,511 to Terry, Jr.; U.S. Pat. No. 5,095,905 to Klepinski; U.S. Pat. No. 5,143,067 to Rise et al.; U.S. Pat. No. 5,215,089 to Baker, Jr.; U.S. Pat. No. 5,251,634 to Weinberg; U.S. Pat. No. 5,282,468 to Klepinski; U.S. Pat. No. 5,351,394 to Weinberg; U.S. Pat. No. 5,358,514 to Schulman et al., U.S. Pat. No. 5,501,201 to Grill, Jr. et al.; U.S. Pat. No. 5,531,778 to Maschino et al.; U.S. Pat. No. 5,689,877 to Grill, Jr. et al.; U.S. Pat. No. 5,741,319 to Woloszko et al.; U.S. Pat. No. 5,964,702 to Grill, Jr. et al.; and U.S. Pat. No. 6,308,105 to Duysens et al.

In particular, helical electrodes intended for use on peripheral nerves are disclosed in detail in U.S. Pat. No. 4,573,481 to Bullara and U.S. Pat. No. 4,920,979 to Bullara, both of which are incorporated in their entirety by this reference.

However, there remains a need for an improved electrode. There is a particular need for an electrode that can be configured so that the degree of excitation of the nerve can be controlled so that either all axons within the nerve or a subpopulation of axons within the nerve can be activated by electrical stimulation.

### SUMMARY OF THE INVENTION

Briefly stated, the invention comprises an electrode assembly for surgical implantation on a peripheral nerve, the assembly including a helically formed supporting matrix. A conductive electrode (preferably made of activated iridium) is secured to an inner, side, or exterior surface of the helical matrix, and a connection means is secured to the electrode and extends from the matrix. Preferably, the electrode is partially embedded in the matrix so only a bare electrode surface facing the axis of the helical matrix is exposed.

In a preferred form the surface of the exposed electrode face is roughened to increase the effective area of the electrode surface. The number of individual electrodes in the assembly is dictated by the specific form of neurostimulation to be achieved, but the assembly is useful with either single or plural electrodes.

In general, one embodiment of the invention comprises an electrode assembly for surgical implantation on a nerve of the peripheral nervous system comprising:

(1) a flexible helically formed supporting matrix of dielectric material;

(2) a flexible conductive electrode secured to the surface of the matrix, the electrode having front and rear surfaces and side edges, the front surface being exposed and not covered by the matrix, the electrode occupying only a portion of the cross-sectional periphery of the matrix; and

(3) at least one flexible connector connected to the electrode and extending from the matrix.

In this embodiment, the matrix and electrode generally form a multi-turn hollow helix of a selected pitch. The helix has a free end and is without a supporting core. The turns of the helix are resiliently movable with respect to each other to enable the helix to be wrapped around an unsevered nerve, such as a nerve of the peripheral nervous system. The helix has a central passage therethrough of size and configuration generally conforming to the external size and configuration of the nerve around which the electrode assembly is to be wrapped. The pitch is selected such that either the entire nerve or any portion of it extending radially inward from its surface forming an annulus can be electrically stimulated. This allows the activation of either substantially all axons or a subpopulation of axons such as those axons located near the periphery of the nerve, according to the helical pitch chosen. The size of the subpopulation is determined by the amplitude of the stimulus current.

In an alternative embodiment, a plurality of spaced-apart flexible conductor ribbon electrodes is used. This embodiment comprises:

(1) a flexible supporting matrix formed substantially in the shape of a spiral helix; and

(2) a plurality of spaced-apart flexible conductor ribbon electrodes secured to and arrayed along an inner surface of the matrix, each electrode occupying only a portion of the cross-sectional periphery of the matrix and having a separate flexible conductor extending therefrom.

In this embodiment, the conductors are embedded in the matrix to extend from the respective ribbon electrodes to an end of the matrix. In this embodiment, the matrix and electrode generally form a multi-turn hollow helix of a selected pitch. The helix has a free end and is without a supporting core. The turns of the helix are resiliently movable with respect to each other as described above. The helix has a central passage therethrough of size configuration generally conformed to the external size and configuration of the nerve around which the electrode assembly is to be wrapped. The pitch of the helix is selected such that the entire nerve or a portion of it extending radially inward from its surface to form an annulus can be electrically stimulated as described above.

In these and other embodiments, the pitch of the helix or helices can be selected such that substantially the entire nerve is stimulated so that substantially all axons of the nerve are easily activated. Alternatively, the pitch can be selected such that a portion of the nerve extending radially inward from its surface forming an annulus is more easily stimulated. This allows for a more orderly and predictable recruitment of the axon population, as the stimulus current is increased.

In another embodiment, the helix has multiple turns. In general, this embodiment comprises:

(1) a flexible helically formed supporting matrix of dielectric material;

(2) a flexible conductive electrode secured to the surface of the matrix, the electrode having front and rear surfaces and side edges, the front surface being exposed and not covered by the matrix, the electrode occupying only a portion of the cross-sectional periphery of the matrix; and

(3) at least one flexible connector connected to the electrode and extending from the matrix.

The matrix and electrode generally form a multi-turn hollow helix of a selected pitch and with a selected number of helical turns of the electrode. The helix has a free end and is without a supporting core, as described above. The turns of the helix are resiliently movable with respect to each other to enable the helix to be wrapped around an unsevered nerve, as described above. The helix has a central passage therethrough of size and configuration generally conforming to the external size and configuration of the nerve around which the electrode assembly is to be wrapped.

In this embodiment, the pitch of the helix and the number of helical turns are selected such that the entire nerve or any portion of it extending radially inward from the surface forming an annulus can be electrically stimulated to activate substantially all axons or a subpopulation of axons.

In yet another embodiment, a plurality of electrodes is used and each electrode includes a multi-turn helix. In general, this embodiment comprises:

(1) a flexible supporting matrix formed substantially in the shape of a spiral helix; and

(2) a plurality of spaced-apart flexible conductor ribbon electrodes secured to and arranged along an inner surface of the matrix, each electrode occupying only a portion of the cross-sectional periphery of the matrix and having a separate flexible conductor extending therefrom.

In this embodiment, the conductors are embedded in the matrix to extend from the respective ribbon electrodes to an end of the matrix.

In this embodiment, the matrix and electrode generally form a multi-turn hollow helix of a selected pitch and with a selected number of helical turns of each electrode. The helix has a free end and is without a supporting core. The turns of the helix are resiliently movable with respect to each other to enable the helix to be wrapped around an unsevered nerve, as described above. The helix has a central passage therethrough of size and configuration generally conforming to the external size and configuration of the nerve around which the electrode assembly is to be wrapped. The pitch of the helix and the number of helical turns of each electrode are selected such that substantially the entire nerve or a portion of it extending radially inward from the surface forming an annulus can be selectively stimulated to activate substantially all axons or a subpopulation of axons as desired.

Another embodiment of the invention is a device for treating a particular condition by electrical stimulation of a particular nerve. The condition and treatment can be one of the following:

(1) treatment of obstructive sleep apnea by stimulation of the glossopharyngeal nerve;

(2) treatment of epilepsy by stimulation of the vagus nerve;

(3) producing diaphragm contractions by electrical stimulation of the phrenic nerve;

(4) restoring hand function by stimulation of a peripheral nerve in the upper extremities; and

(5) regulating gait by electrical stimulation of a peripheral nerve in the lower extremities.

In general, such a device comprises:

(1) a helical electrode assembly comprising:

(a) a flexible helically formed supporting matrix of dielectric material;

(b) at least one flexible conductive electrode secured to the surface of the matrix, the electrode having front and rear surfaces and side edges, the front surface being exposed and not covered by the matrix, the electrode occupying only a portion of the cross-sectional periphery of the matrix; and

(c) at least one flexible connector connected to the at least one electrode and extending from the matrix; the matrix and at least one electrode generally forming a multi-turn hollow helix of a selected pitch and with a selected number of helical turns of each electrode, the helix having a free end and without a supporting core, the turns of the helix being resiliently movable with respect to each other to enable the helix to be wrapped around the unsevered nerve to be treated, the helix having a central passage therethrough of size configuration generally conforming to the external size configuration of the nerve to be treated, the pitch of the helix and the number of helical turns being selected such that the entire nerve to be treated or any portion of it extending radially inward from the surface forming an annulus can be electrically stimulated to activate substantially all axons or a subpopulation of axons of the nerve;

(2) a controllable current or voltage source in electrical contact with the at least one flexible connector; and

(3) a controller for controlling the current or voltage source so that an electric field is created by at least one electrode to treat the specified condition.

### BRIEF DESCRIPTION OF THE DRAWINGS

FIG. 1 is a diagram of an embodiment of an electrode assembly according to the present invention that has one electrode with a plurality of helical turns;

FIG. 2 is a diagram of another embodiment of an electrode assembly according to the present invention that has more than one electrode, each with a plurality of helical turns;

FIG. 3 is a view of an electrode assembly according to the invention as mounted on a peripheral nerve and coupled to an implanted neurostimulator circuit (shown in phantom line);

FIG. 4 is a plan view of the electrode assembly when unwound from the helical form into a flat strip;

FIG. 5 is a sectional view on line 3-3 of FIG. 4;

FIG. 6 is a sectional view on line 4-4 of FIG. 4;

FIG. 7 is a sectional view on line 5-5 of FIG. 4;

FIG. 8 is a view of a mandrel;

FIG. 9 is a view of a portion of the electrode assembly wound on the mandrel;

FIG. 10 is a schematic diagram of a single-electrode system;

FIG. 11 is a schematic diagram of a triple-electrode system;

FIG. 12 is a view of an alternative electrode assembly having opposite rewound spiral segments;

FIG. 13 is a plan view of a pair of electrodes for another embodiment of the invention;

FIG. 14 is a view similar to FIG. 13 after connecting wires to the electrodes has been attached;

FIG. 15 is a pictorial view of a monopolar electrode assembly that is another embodiment of the invention with closely spaced helical portions;

FIG. 16 is a view similar to FIG. 15, but showing a multipolar electrode assembly with more widely spaced helical portions;

FIG. 17 is a plan view of the electrode assembly of FIG. 16 as distorted into an unwound flat shape;

FIG. 18 is a view similar to FIG. 16, and showing the flattened configuration of the FIG. 15 assembly;

FIG. 19 is a pictoral view of an installation tool for the assembly of FIG. 15;

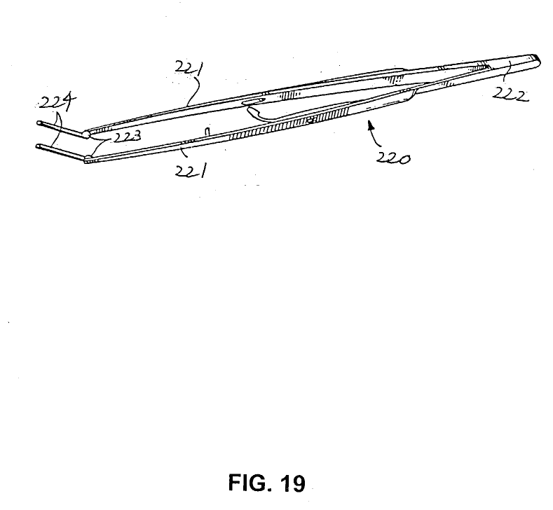

FIG. 20 shows the installation tool fitted and expanded within the electrode assembly to open the helical portions which are positioned for placement over a nerve;

FIG. 21 shows the tool being removed after placement of the assembly around the nerve;

FIG. 22 is an end view of a portion of the assembly as expanded by the tool over the nerve;

FIG. 23 is an enlarged section through one of the helical portions;

FIG. 24 is a diagram of the structure of a typical peripheral nerve;

FIG. 25 is a photograph of an assembly according to the present invention installed on a peripheral nerve;

FIG. 26 is a diagram showing the locations at which current density was modeled using a finite-element model;

FIG. 27 is a graph showing the relative threshold of an axon plotted against the percentage of distance from the center of the nerve for a one-turn helical electrode with a monopolar configuration with the helix starting 90° clockwise of the plane of computation for pitches from 0 to 3, including an example with the pitch equal to 3 and two full helical turns; the pitch of the helix is the distance that the helix advances along its nerve's longitudinal direction for each complete turn; thus, with a pitch of 2, the helix advances by 2R with each complete encirclement (R is the radius of the nerve); and

FIG. 28 is a graph showing the relative threshold plotted against the percentage of distance from the center of the nerve for a one-turn helical electrode with a monopolar configuration where the helix starts at 180° clockwise of the plane of computation for pitches of 0 to 3.

### DESCRIPTION OF THE PREFERRED EMBODIMENT

By adjusting the pitch of the helix and the number of helical turns, the difference between the field gradient at the center and the periphery of the nerve can be adjusted. The greater this field difference, the greater the ability to excite the axons lying near the periphery of the nerve while not exciting the axons at the center of the nerve. The more shallow this difference in field gradient, the less the degree of differentiation in activation. Thus, by increasing the pitch of the helix and the number of helical turns, one can configure such electrodes to achieve an orderly recruitment of the axon population as the stimulus current is incresed. It is notable that this flexibility can be achieved with only a single channel of stimulation, thus avoiding the added complexity and cost of a multi-channel system. Conversely, by decreasing the pitch of the electrodes, one can configure such electrodes to stimulate substantially all the axons of the nerve with a shallower difference in field gradient between the field gradient at the center and at the periphery of the nerve. This is shown in Example 1.

Accordingly, in general, one embodiment of the invention comprises an electrode assembly for surgical implantation on a nerve of the peripheral nervous system comprising:

(1) a flexible helically formed supporting matrix of dielectric material;

(2) a flexible conductive electrode secured to the surface of the matrix, the electrode having front and rear surfaces and side edges, the front surface being exposed and not covered by the matrix, the electrode occupying only a portion of the cross-sectional periphery of the matrix; and

(3) at least one flexible connector connected to the electrode and extending from the matrix.

In this embodiment, the matrix and electrode generally form a multi-turn hollow helix of a selected pitch. The helix has a free end and is without a supporting core. The turns of the helix are resiliently movable with respect to each other to enable the helix to be wrapped around an unsevered nerve, such as a nerve of the peripheral nervous system. The helix has a central passage therethrough of size and configuration generally conforming to the external size and configuration of the nerve around which the electrode assembly is to be wrapped. The pitch is selected such that either the entire nerve or any portion of it extending radially inward from its surface forming an annulus can be electrically stimulated. This allows the easy activation of either substantially all axons or a subpopulation of axons such as those axons located near the periphery of the nerve, according to the helical pitch chosen. The size of the subpopulation is determined by the amplitude of the stimulus current. As demonstrated below in Example 1, as the pitch of the helix increases or as the number of helical turns increases, there is a greater difference between the field gradient at the center and at the periphery of the nerve. This allows activation of the axons located near the periphery with a relatively low voltage applied to the electrode. The pitch of the helix is defined, as is generally known in the art, as the distance along the helix for a 360-degree rotation of the helix around the central axis.

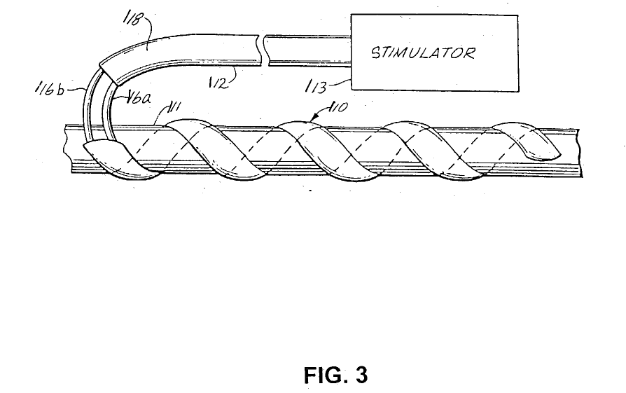

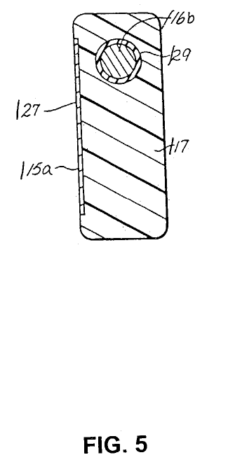

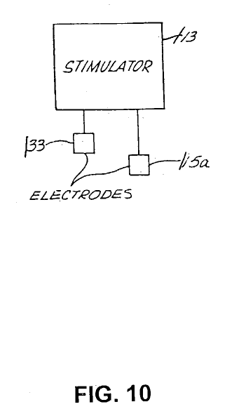

Examples of this embodiment are depicted below in FIGS. 3-14.

A number of alternatives of this embodiment are possible. In one alternative, the electrode is partially embedded in the matrix. In this alternative, typically the matrix extends over the rear surface and side edges of the electrode. In this alternative, typically, also, one end of the electrode is folded rearwardly, and the folded end is fully embedded in the matrix. Furthermore, in this alternative, typically the connection means is embedded in the matrix between the electrode and an end of the matrix.

In this embodiment, and other embodiments, preferably the front surface of the electrode is roughened to increase the effective area of the surface to improve the contact between the electrode and the nerve. Typically, the degree of surface roughening increases the effective surface area by a factor of at least 10 as compared with a perfectly smooth surface. Preferably, the degree of surface roughening increases the effective surface area by a factor of at least about 20 as compared with a perfectly smooth surface.

The flexible connector can include a flexible stranded wire. The flexible connector can further include a conductor ribbon wrapped around an end of the wire and welded thereto to form a flattened connection tab for attachment to the electrode.

Preferably, the electrode is activated iridium.

Typically, the flexible supporting matrix is silicone.

In an alternative embodiment, a plurality of spaced-apart flexible conductor ribbon electrodes is used. This embodiment comprises:

(1) a flexible supporting matrix formed substantially in the shape of a spiral helix; and

(2) a plurality of spaced-apart flexible conductor ribbon electrodes secured to and arrayed along an inner surface of the matrix, each electrode occupying only a portion of the cross-sectional periphery of the matrix and having a separate flexible conductor extending therefrom.

In this embodiment, the conductors are embedded in the matrix to extend from the respective ribbon electrodes to an end of the matrix. In this embodiment, the matrix and electrode generally form a multi-turn hollow helix of a selected pitch. The helix has a free end and is without a supporting core. The turns of the helix are resiliently movable with respect to each other as described above. The helix has a central passage therethrough of size configuration generally conformed to the external size and configuration of the nerve around which the electrode assembly is to be wrapped. The pitch of the helix is selected such that the entire nerve or a portion of it extending radially inward from its surface to form an annulus can be electrically stimulated as described above.

In these and other embodiments, the pitch of the helix or helices can be selected such that substantially the entire nerve is stimulated so that substantially all axons of the nerve are activated, at nearly the same threshold. Alternatively, the pitch can be selected such that a portion of the nerve extending radially inward from its surface forming an annulus is stimulated so that a subpopulation of axons is activated, the size of the subpopulation being determined by the amplitude of the applied current.

In one alternative, when the pitch is selected so that a subpopulation of axons is activated, the assembly is configured to stimulate the glossopharyngeal nerve to extend the tongue to treat obstructive sleep apnea. In other alternatives, the assembly is configured to stimulate the vagus nerve to treat epilepsy. The assembly can be configured to stimulate other peripheral nerves.

In another embodiment, the helix has multiple turns. In general, this embodiment comprises:

(1) a flexible helically formed supporting matrix of dielectric material;

(2) a flexible conductive electrode secured to the surface of the matrix, the electrode having front and rear surfaces and side edges, the front surface being exposed and not covered by the matrix, the electrode occupying only a portion of the cross-sectional periphery of the matrix; and

(3) at least one flexible connector connected to the electrode and extending from the matrix.

The matrix and electrode generally form a multi-turn hollow helix of a selected pitch and with a selected number of helical turns of the electrode. The helix has a free end and is without a supporting core, as described above. The turns of the helix are resiliently movable with respect to each other to enable the helix to be wrapped around an unsevered nerve, as described above. The helix has a central passage therethrough of size and configuration generally conforming to the external size and configuration of the nerve around which the electrode assembly is to be wrapped.

In this embodiment, the pitch of the helix and the number of helical turns are selected such that the entire nerve or any portion of it extending radially inward from the surface forming an annulus can be electrically stimulated to activate substantially all axons or a subpopulation of axons.

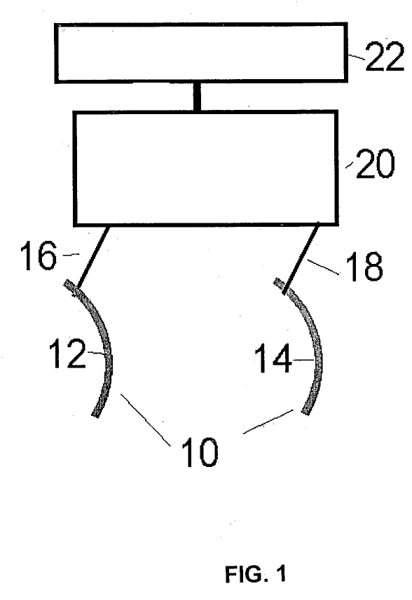

In one alternative of this embodiment, each turn of the plurality of helical turns is electrically isolated and connected to a separate connector controlled by a switch so that voltage can be applied to one or more of the plurality of the helical turns as controlled by the switch. This alternative of the embodiment is shown schematically in FIG. 1. In FIG. 1, the electrode 10 is shown for illustrative purposes as having two helical turns, a first helical turn 12 and a second helical turn 14; of course, in an actual electrode, more helical turns are possible. The first helical turn 12 and the second helical turn 14 are electrically isolated so that a voltage applied to the first helical turn 12 is not applied to the second helical turn 14 and vice versa. The first helical turn 12 is connected electrically to a first connector 16. The second helical turn 14 is connected electrically to a second connector 18. The first and second connectors 16 and 18 are connected electrically to a switch 20 and voltage source 22 so that voltage can be applied independently to one or more of the helical turns.

In yet another embodiment, a plurality of electrodes is used and each electrode includes a multi-turn helix. In general, this embodiment comprises:

(1) a flexible supporting matrix formed substantially in the shape of a spiral helix; and

(2) a plurality of spaced-apart flexible conductor ribbon electrodes secured to and arranged along an inner surface of the matrix, each electrode occupying only a portion of the cross-sectional periphery of the matrix and having a separate flexible conductor extending therefrom.

In this embodiment, the conductors are embedded in the matrix to extend from the respective ribbon electrodes to an end of the matrix.

In this embodiment, the matrix and electrode generally form a multi-turn hollow helix of a selected pitch and with a selected number of helical turns of each electrode. The helix has a free end and is without a supporting core. The turns of the helix are resiliently movable with respect to each other to enable the helix to be wrapped around an unsevered nerve, as described above. The helix has a central passage therethrough of size and configuration generally conforming to the external size and configuration of the nerve around which the electrode assembly is to be wrapped. The pitch of the helix and the number of helical turns of each electrode are selected such that substantially the entire nerve or a portion of it extending radially inward from the surface forming an annulus can be selectively stimulated to activate substantially all axons or a subpopulation of axons as desired.

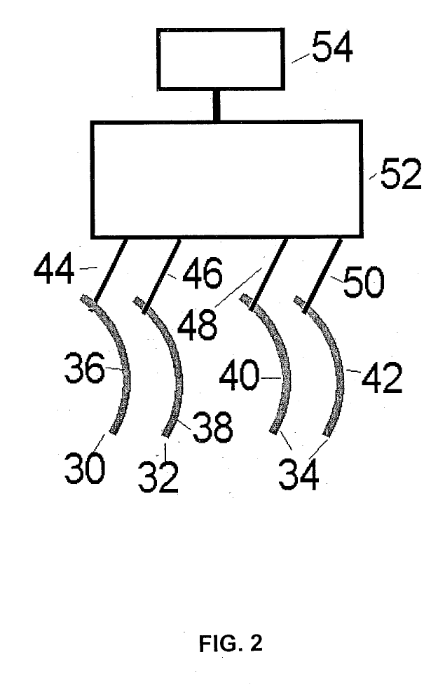

In another alternative of this embodiment, each turn of the plurality of helical turns for each electrode is electrically isolated and connected to a separate connector controlled by a switch so that voltage can be applied to one or more of the plurality of the helical turns for each electrode as controlled by the switch. This alternative of the embodiment is shown schematically in FIG. 2. In FIG. 2, the electrode assembly 30 comprises a first electrode 32 and a second electrode 34. Two electrodes are shown for illustrative purposes, but more than two electrodes can be used in an actual assembly. In FIG. 2, the first and second electrodes 32 and 34 are shown for illustrative purposes as having each two helical turns, first helical turns 36 for the first electrode 32 and 38 for the second electrode 34 and second helical turns 40 for the first electrode 32 and 42 for the second electrode 34; of course, in an actual assembly, more helical turns are possible for each electrode. The first helical turns 36 and 38 and the second helical turns 40 and 42 are electrically isolated so that a voltage applied to the first helical turns 36 and 38 is not applied to the second helical turns 40 and 42 and vice versa. The first helical turns 36 and 38 are connected electrically to first connectors 44 and 46. The second helical turns 40 and 42 are connected electrically to second connectors 48 and 50. The first connectors 44 and 46 and the second connectors 48 and 50 are connected electrically to a switch 52 and voltage source 54 so that voltage can be applied independently to one or more of the helical turns for each electrode.

As indicated below in Example 1, in general, with a constant pitch, the greater the number of helical turns of the electrode, the greater the difference between the field gradient at the center and at the periphery of the nerve, with the field gradient always being greater at the periphery of the nerve. Thus, both the pitch of the helix and the number of helical turns can be adjusted to yield the desired curve for differential stimulation of the axons located closer to the periphery of the nerve and those located in the center of the nerve so that substantially all of the axons or a subpopulation of the axons can be activated.

In addition to the use of electrode assemblies according to the present invention for the stimulation of the glossopharyngeal nerve to treat obstructive sleep apnea and the vagus nerve to treat epilepsy, such electrode assemblies can also be used to stimulate the auditory nerves to restore hearing (G. M. Clark et al., “Cochlear Prostheses”, Churchill-Livingston, N.Y., 1990). The phrenic nerve of patients with high-level spinal cord injury can be stimulated to produce diaphragm contractions and restore ventilation (W. W. L. Glenn et al., “Ventilatory Support by Pacing of the Conditioned Diaphragm in Quadraplegia,” N. Engl. J. Med. 310:1150 (1984)). Similarly, electric stimulation of paralyzed patients can restore partial function of both the upper extremities for hand function (P. H. Peckham et al., “Restoration of Function of Control by Electrical Stimulation in the Upper Extremity of the Quadraplegic Patient,” J. Bone Joint Surg., 70A:144-148 (1987)) and lower extremities for gait (E. B. Marsolais & R. Kobetic, “Development of a Practical Electrical Stimulation System for Restoring Gait in the Paralyzed Patient,” Clin. Othrop., 233:64 (1988)). Electrode assemblies according to the present invention can also be adapted for stimulation of other nerves, both in the peripheral nervous system and, with appropriate design constraints, in the central nervous system.

In general, embodiments are as described below.

FIG. 3 shows a helical electrode assembly 110 according to the invention, installed on a peripheral nerve 111. A cable 112 connects the assembly to an implantable neurostimulator or bioelectronic receiver 113 arranged to receive command signals transmitted from outside the body. The receiver sends current signals to the electrode assembly in the nerve in response to the command signals. Bioelectronic receivers of various designs are known in the art and are not discussed in detail here.

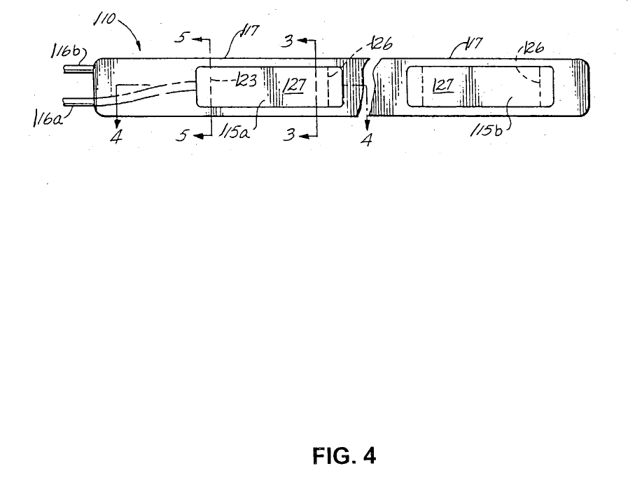

Referring to FIGS. 4-6, the assembly (shown as “unwound” into an elongated flat strip) includes one or more electrodes 115, connecting wire 116 secured to each electrode, and a strip-like supporting matrix 117 which holds and positions the wires and electrodes. The electrodes have selected helical pitch and a selected number of helical turns, as described above. Depending on the physiological function to be accomplished, the assembly may have one, two, three or more electrodes. The configuration shown in the drawings has two electrodes 115a and 115b, and the associated wires are designated as 116a and 116b.

Long-term implantation is desired in many applications of the electrode assembly, and it is important that the assembly components be capable of sterilization and made of materials which are acceptable to the body. Preferably, the electrodes are made of activated iridium, but platinum or rhodium (or alloys of these metals) are alternative materials. The supporting matrix is a moldable medical-grade silicone, such as sold under the trademark Dow-Corning 905 (Adhesive-type A) or alternating layers of this material in Dow-Corning 382 medical-grade silicone. The latter material enables better control of matrix resiliency, and it bonds well to the Dow-Corning 905 material.

The use of activated pure iridium in neural-stimulation electrodes is described in detail in Robblee et al., J. Electrochem. Soc. 130:731-733 (1983). The iridium ribbon material is typically activated by formation of a high-valence iridium oxide surface layer on the ribbon. Charge injection is facilitated by oxidation and reduction of the surface layer, and undesired erosion of the underlying pure metal is reduced to eliminate it.

Conducting wires are preferably highly flexible stranded conductors, and a wire formed of about 45 strands of very fine (0.0005 to 0.006 inch) (0.00127 to 0.00152 centimeters))diameter stainless steel wire is satisfactory. Between the electrode assembly and the bioelectronic receiver, each wire has an insulating jacket 118 (FIG. 3) of medical-grade silicone as mentioned above.

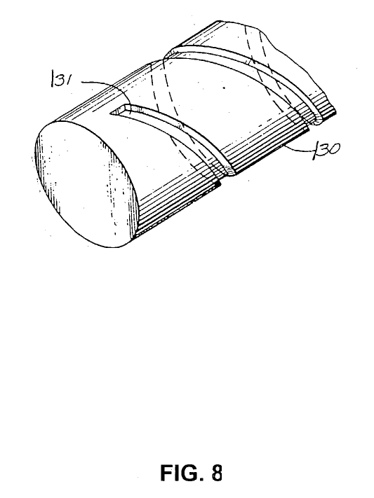

In making prototype models of the assembly, an initial step is to strip the insulating jacket from the end of each wire 116 and then to wrap the bare wire spirally or helically around a small-diameter (e.g., in the range of about 2-5 mm, depending on the size of the nerve for which the assembly is intended) cylindrical mandrel, as shown in FIG. 8. The wire and mandrel are then heated in a vacuum furnace to about 800° C. to anneal and stress relieve the strands, and to give the wire a permanent spiral configuration. This step is completed under high vacuum to avoid oxidation which can reduce strand strength and interfere with subsequent electrode welding.

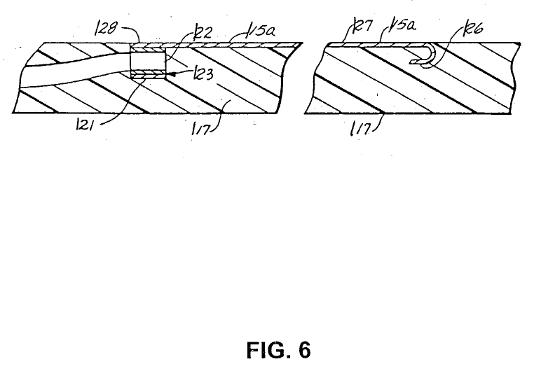

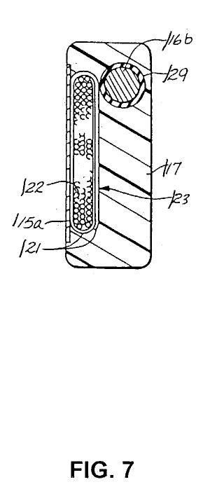

With the exposed end of the wire now set in a corkscrew shape, a narrow (about 0.25-0.4 mm) activated-iridium ribbon 121 is wrapped around tip 122 of the wire end for about 1.5 turns as shown in FIGS. 6 and 7. The ribbon is resistance welded to the wire tip of the power-electrode rotor which flattens the ribbon and stranded wire into a pad or tab 123 which can be readily welded to the associated electrode 115.

Each electrode 115 is initially formed as a generally rectangular strip of activated iridium ribbon of about 0.0005-inch (0.00127-cm) thickness. In a typical configuration, the ribbon is about 0.75 to 1.0 mm wide and of sufficient length to spiral entirely around the diameter of the selected nerve.

For example, if the nerve on which the electrode assembly is to be implanted is about 2 mm in diameter, and the helical pitch of the electrode is about 3 mm, the electrode length should be at least 7 mm (and preferably 8 or 9 mm as a safety factor). This ensures that the installed electrode wire surrounds the nerve to deliver stimulus signals to the sub-bundles which comprise the main nerve bundle. As indicated above, the helical pitch of the electrode is varied depending upon the application for which the electrode is intended, particularly with respect to whether it is desired to excite substantially all of the axons of the nerve or a subpopulation of axons lying near the periphery of the nerve in an annulus. The number of helical turns is also varied.

As shown in FIG. 6, one end 126 (remote to the end to be soldered to tab 123) of the electrode ribbon is folded back on itself, the direction of fold being away from a front surface 127 which will face and contact the nerve when the assembly is implanted. An opposite end 128 of the ribbon is then positioned over tab 123 of associated connecting wire 116, and the ribbon and tab are resistance welded together.

An important step in completing preparation of the electrode is to increase the surface roughness of front surface 127. While the surface roughening can be done in a number of ways, a simple and effective technique is to peen or “sandblast” surface 127 with very small glass beads, or preferably with salt crystals which can be easily dissolved and removed to ensure electrode cleanliness after the desired degree of roughness is achieved.

The purpose of surface roughening is to increase significantly the effective surface area of the electrode, and ultimately to enable biostimulation of a nerve bundle at a relatively low charge density at the nerve surface. There is considerable work that shows that an excessively high charge density at the nerve surface can cause nerve damage (D. B. McCreery et al., “Damage in Peripheral Nerve from Continuous Electrical Stimulation: Comparison of Two Stimulus Wave Forms,” Med. Biol. Eng. Comput., 30:109 (1992)). Surface roughening can easily increase the effective surface area by a factor of at least about 10, preferably by at least about 20. Surface-area increases in the range of 40 to 50 are believed feasible.

The coiled end of wire 116 and now-secured electrode are next dipped in a bath (preferably agitated by ultrasonic energy) of a liquid epoxy, such as sold under the trademark Epoxylite. When the exposed metal surfaces have been fully coated with liquid epoxy, the wire and electrode are removed from the bath, and all epoxy is removed from front surface 127 which will contact the nerve when the electrode is installed.

The wire and electrode ribbon are then baked to cure the epoxy, and to form a flexible layer of insulating material 29 on the wire and the back surface of the electrode. The specified epoxy material is presently preferred as it bonds well with silicone in the subsequent manufacturing step as described below.

An arbor or mandrel 130 as shown in FIG. 8 is used in completing the electrode assembly. The mandrel has a helical groove 131 formed in its surface, and the groove has a generally rectangular cross-section. The groove is typically about 0.030 to 0.040 inch (0.0762 to 0.102 cm) deep, and is slightly wider than the electrode ribbon being used. The helical pitch of the groove corresponds to the pitch desired in the finished spiral electrode, and the inside diameter of the groove corresponds to the desired inside diameter of the finished electrode.

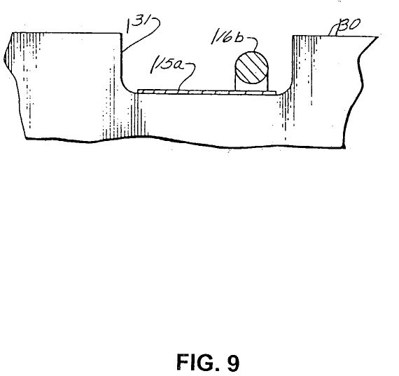

Wires 116a and 116b, and associated electrodes 115a and 115b, are now wound into groove 131 as shown in FIG. 9. Wire 116b is longer than wire 116a, so that the electrodes will be axially spaced apart, preferably by at least about 10 mm. Silicone of the type already described is then deposited in the groove to encapsulate the wires, and to surround the electrodes with the exception of front surfaces 127, which remain exposed. The silicone extends away from the electrode sufficiently far to join and become bonded to insulating jacket 118 on each wire.

Preferably, the mandrel groove is slightly over-filled to provide a softly rounded top surface on the resulting assembly. As shown in FIGS. 4 and 6, the silicone flows into folded end 126 of each electrode to increase the physical bond between resulting supporting matrix 117 and the electrode. The extra width of the mandrel groove enables the matrix to overhang the thin edges of the electrodes to prevent abrasion or cutting of the nerve bundle.

When the silicone matrix is cured, the completed assembly is stripped away from the mandrel, and is ready for use. Surgical implantation is conventional, and the electrode is gently wrapped around the exposed nerve. The wrapping operation starts with the cable end of the matrix and proceeds to the opposite free end of the matrix.

When so installed, the open construction of the helical electrode minimizes interruption of the necessary fluid exchange between the nerve and its surrounding environment. Swelling or edema of the nerve as a result of surgical manipulation is accommodated by a slight “unwinding” of the helical coil to enlarge the coil diameter in response to tissue pressure. Good electrical contact of the electrode and nerve is provided by the gentle springiness of the silicone matrix, and tests have shown that interruptive ingrowth of fatty or connective tissue is significantly reduced as compared to that encountered with cuff-type electrodes.

The helical electrode assembly of the present invention is useful in a variety of ways in association with the peripheral nerve system, and the number and spacing of electrodes, as well as the dimensions of the helix, will vary depending upon the objective to be achieved. As detailed above, and in Example 1 below, the pitch of the helix of the electrode or electrodes, and the number of helical turns can be varied depending upon whether it is desired to excite substantially the entire nerve or a portion of it extending radially inward from the surface of the nerve, forming an annulus. In some cases, a single electrode only is needed, and this arrangement is shown in schematic form in FIG. 8, with the second contact being made by a common or “indifferent” electrode 133, which may be remote from the nerve.

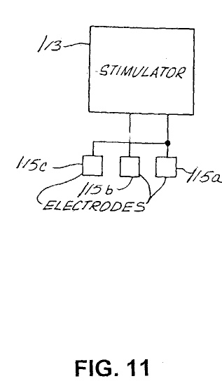

FIG. 11 shows the wiring arrangement of one form of a three-electrode array, which is useful in certain muscle-stimulation applications. Voltage and current levels can vary considerably, but a typical stimulating pulse is in the range of about 15 volts at about 3 milliamperes. As mentioned above, the range of application of the electrode assembly is not limited to muscle stimulation, and blocking of nerve conduction or monitoring of action potentials are other suitable uses.

Depending upon the cross-sectional shape of the nerve to be stimulated, it may be desirable to form the electrode with a generally elliptical (in cross-section) central opening to provide good conformance with the nerve surface. Variations of this type are easily achieved by changing the cross-sectional shape of mandrel 130.

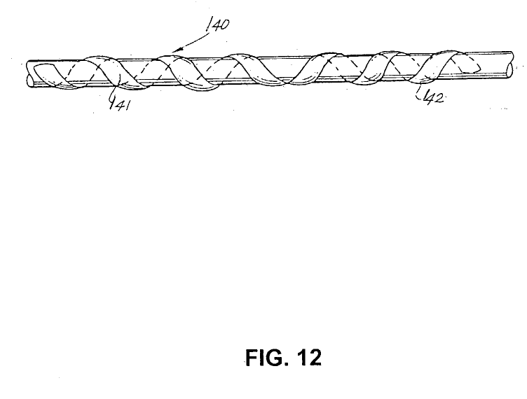

Another alternative arrangement is to form an electrode assembly 140 (FIG. 12) having opposite rewound spiral segments 141 and 142. The oppositely wound segments are believed to be useful in minimizing contraction or migration of the electrode assembly in situations where the associated nerve bundle is subject to active skeletal or muscle movement.

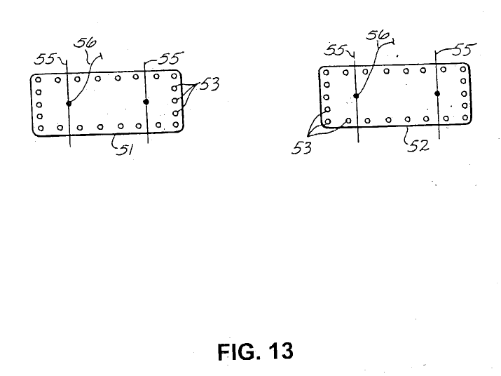

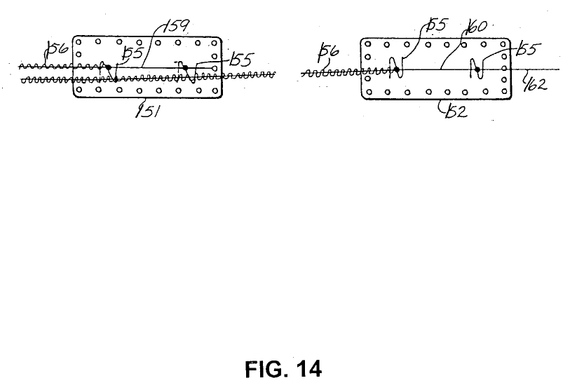

A further alternative electrode configuration is shown in FIGS. 13 and 14, and it again uses a pair of spaced-apart electrodes 151 and 152, which are preferably made of activated iridium. To provide improved adhesion to subsequently applied insulating materials and the silicone matrix, dimples or small holes 153 are preferably formed along the periphery of each generally rectangular electrode.

As shown in FIG. 13, a pair of short spaced-apart tie wires 155 and a long connecting wire 156 are spot-welded to the back surface of each electrode. Both the tie wires 155 and the connecting wires 156 can be 0.002-inch (0.0051-cm) diameter platinum/10% iridium wire. The wires on the back surface of the electrode are then coated with an epoxy-like material which is baked at about 170° C. The front surfaces of the electrodes which will contact the nerve bundle are, of course, left uncoated.

A thread 159 of 5-0 Dacron suture material is then placed on the back of electrode 151, and a longer second thread of the same material is positioned across the rear surfaces of both electrodes 151 and 152. Tie wires 155 are folded over the threads to secure them in place as shown in FIG. 14. Connecting wires 156 are then spiral-wound along the threads to provide a strain-relieved pair of connections to their respective electrodes. A light coating of silicone-type A adhesive is then applied to the spiral-wound wires and allowed to dry.

Thread 159 terminates within the perimeter of electrode 151, but thread 160 extends beyond electrode 152 by several centimeters, as shown in FIG. 14. This tag end 162 of thread 160 provides a “tail” which can be gripped by the surgeon to gently wind the spiral electrode around a nerve bundle.

The remaining fabrication steps are the same as already described with respect to assembly 110. That is, the electrodes and connecting wires are wrapped around a spiral mandrel, and covered with a supporting matrix of silicone material to form the complete spiral electrode assembly. Connecting wires 156 extend from the completed spiral assembly for connection to wires in the connecting cable (not shown).

In some applications of the spiral electrode assembly of this invention on the peripheral nervous system (in, for example, the arms or legs), the nerve may be adjacent to a major muscle or other body structure which presses the electrodes against the nerve with considerable pressure in certain body positions. In other body positions, this pressure is released, perhaps leaving a small fluid-filled space between the electrode and nerve surface. These electrode-nerve spacing variations can sometimes present a problem in that the stimulation effectiveness of a given electrical signal is significantly influenced by spacing.

In such applications, it is unnecessary that the electrodes be located on the inside diameter of the supporting spiral matrix, and the electrodes are instead positioned on the side surfaces (or even the top surface) of the spiral matrix. Such side- or top-mounted electrodes should extend entirely around the nerve for the reasons described above, and the electrodes are preferably again formed of activated iridium foil or wire. This arrangement sometimes requires a somewhat higher stimulating signal, but small spacing variations due to muscle or skeletal movement have little effect due to the larger average separation of the electrode and nerve surface.

When a side- or top-mounted electrode array is used, the electrode assembly is preferably covered with a loose-fitting insulating sheath or cuff to minimize outwardly directed charge injection away from the nerve into overlying tissue. The insulating sheath tends to confine charge injection to the target nerve, and reduces unwanted stimulation of adjacent tissue.

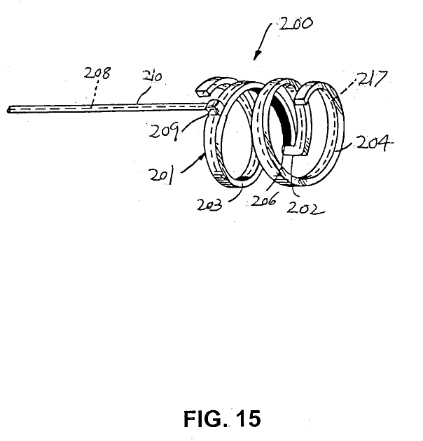

In another embodiment of an electrode assembly according to the invention, two helical portions extend away from a central junction or bridge portion. This embodiment is shown in FIG. 15. The assembly 200 includes a preformed resilient and insulating matrix 201 having a central junction or bridge portion 202. Two helical portions 203 and 204 extend integrally from the bridge portion, and the helical portions advance away from the bridge portion in opposite directions. Each helical portion extends around at least 360°, and preferably around 420° to about 540°. The pitch of the helical turns typically is small, and adjacent windings are typically spaced apart by less than the axial width of the helical-portion turns, and preferably by about one-third the turn axial width. However, the pitch of the helix can be adjusted as described above to control the degree of excitation of the nerve, i.e., to excite substantially all the axons or a subpopulation of axons.

Assembly 200 is a monopolar configuration having a single conductive electrode 206 secured on the inner surface of helical portion 203. The electrode is a thin and flexible metal ribbon, as described above, embedded in the inner matrix surface, but with the inwardly facing surface of the ribbon fully exposed for electrical contact with the nerve. Depending upon the type of nerve stimulation desired, the electrode may extend around a full turn of the helical portion, or somewhat less than a full turn, as shown in FIG. 15.

A connection means for coupling the electrode to a source (not shown) of electrical signals is formed by a flexible multi-strand wire 208 welded to the outer embedded surface of the electrode. The wire extends radially outward from the electrical, with a small button or dimple 209 integrally formed with helical portion 203. The wire is bent 90° within the dimple to extend parallel to the central axis of the helix, and is insulated by a surrounding tubular jacket 210 joined to a dimple.

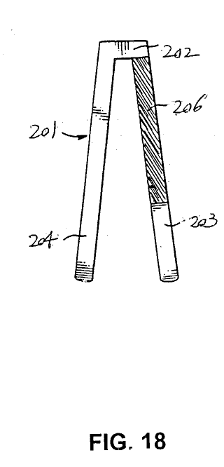

FIG. 18 shows the inner surface of assembly 201 unwound into a flat configuration. This view is provided only for clarifying the assembly structure, and the assembly is not constructed in flat form, nor is it normally distorted or unrolled to this condition during manufacture or use.

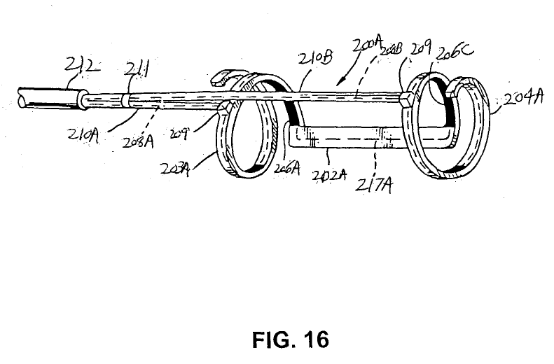

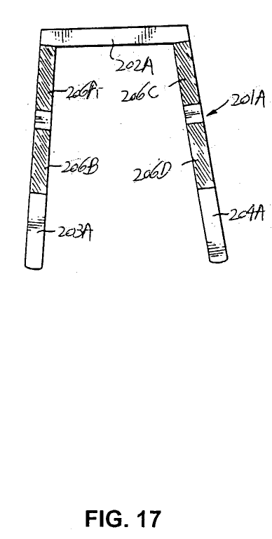

The electrode configuration and the spacing of the helical portions can be varied according to the planned nerve-stimulation program, as described above. A typical variation is shown in FIGS. 16 and 17 as an electrode assembly 200A having a matrix 201A. In this variation of this embodiment, bridge portion 202A is significantly lengthened to increase the axial spacing of oppositely directed helical portions 203A and 204A.

Both of these helical portions are provided with a pair of conductive electrodes 206A-B and 206C-D, and the electrodes of each pair can be driven by a double lead cable 208A (and 208B for electrodes 206C-D), depending on the planned nerve-stimulation protocol. The insulated lead wire jackets 210A and 210B are preferably joined by a drop of adhesive 211 and fitted into a surrounding tubular jacket 212 as they extend away from the assembly. Jacket 212 limits the bending radius of the connecting wire, and helps to prevent kinking, warp hardening, and possible eventual breakage of wire strands during body movement.

The inside diameter of the helical portions is selected to be a close or very gently compressive fit on the nerve to be stimulated. Most peripheral nerves which are candidates for electrical stimulation have outside diameters in the range of about 1.0 to 7.0 mm, and this accordingly establishes the range of typical inside diameters of the helical portions. When electrodes are provided in both helical portions, bridge portion 202A will typically have an axial length in the range of 7-10 mm (but shorter or longer dimensions can be used) for effective stimulation and good evoked response at low power levels.

The supportive matrix of the assembly is preferably formed by a ribbon of medical-grade silicone elastomer, and an acceptable and commercially available uncured formulation is Dow-Corning MDX4-4210. The connecting wire should have high flexibility and integrity, and a teflon-coated 25-strand stainless steel wire in a silicone-rubber jacket is satisfactory. The electrodes are preferably thin and high-purity annealed platinum ribbons about 1 mm in width and 0.025 mm thick for good flexibility. The ribbon is preferably surface-roughened (abrasion with 25 micrometer diamond abrasive is a suitable technique) to increase the effective area of the nerve-contacting phase, and to enable mechanical bonding with the matrix material.

Prototype electrode assemblies are made by methods disclosed in the art, such as those disclosed in U.S. Pat. No. 4,573,481.

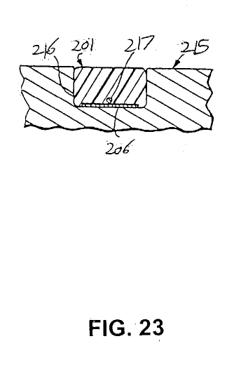

Briefly, and with reference to FIG. 23, an arbor or mandrel 215 is provided with a helical groove 216 corresponding in dimension to the desired geometry of the matrix. Each electrode 206 is fitted against the base of the groove, and is securely positioned and pressed against the groove base by a tightly wrapped strand 207 of 5-0 Dacron suture material. Intimate contact of the electrode against the mandrel is important to prevent any flow of silicone elastomer between the facing surfaces. Wire 208 is prewelded to the radially outer surface of the electrode, and the joint is insulated with an epoxy material, such as sold under the trademark Epoxylite.

The liquid components of the silicone elastomer are then mixed and degassed to eliminate bubbles, and the elastomer is applied to the mandrel to fill groove 216 which defines the bridge and helical portions of the matrix. The elastomer is cured by heating to complete the formation of the assembly, which is then gently stripped away from the mandrel. It is important that the cured matrix have good shape retention combined with high flexibility and resiliency, and the aforementioned silicone elastomer satisfies these requirements.

In a typical configuration of this embodiment, matrix 201 has a generally rectangular cross-section, with an axial width of about 1.2 mm, and a radial thickness in the range of about 0.6 to 0.8 mm. The lower end of the thickness range is used for electrode assemblies intended for nerves of small diameter, the larger thicknesses are selected for larger nerves to maintain approximately constant radial stiffness of the helical turns.

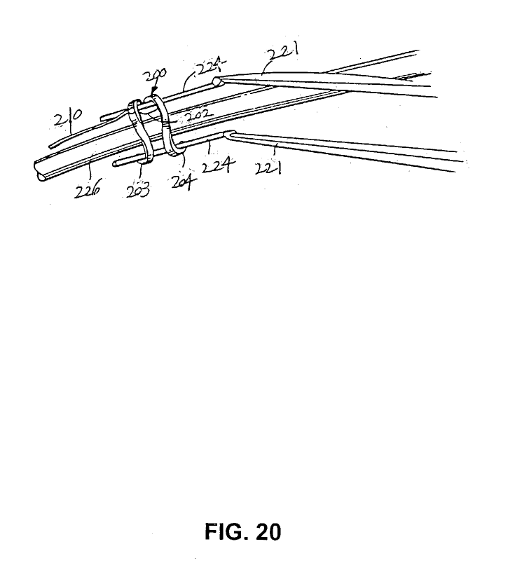

Installation tool 220 for the electrode assembly is shown in FIG. 20, and the tool is a modified surgical tweezer having a pair of legs 221 extending from a base junction 222 to tips 223. The legs are normally biased apart to separate tips 223, but the tips can be brought together by squeezing the tweezer in conventional fashion.

The tweezer is modified by the addition of a pair of tines or pins 224, each of which is welded or brazed to a respective leg tip 223. The pins are parallel, and extend at about 450 from the longitudinal axes of the legs. This angulation permits the pins to be oriented parallel to a nerve as described below, with the tweezer body extending upwardly away from the nerve for manipulation by the surgeon and good visibility of the electrode assembly. The pins are longer than the axial dimension of the electrode assembly to be installed, and are typically about 18 mm long.

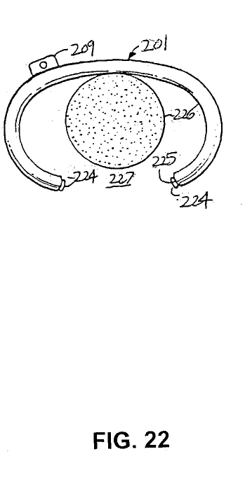

Although the pins may have a simple circular cross-section, a preferred trough-shaped cross-section is shown in FIG. 22. The concave side of this cross-section forms a shallow depression or seat 225 to receive the circumferential ends of the helical portions (or the corresponding end of the matrix bridge portion), and thus to support the opened electrode during the installation procedure described below. The trough-shaped cross-section also minimizes pin size for fitting within helical assemblies of very small inside diameter.

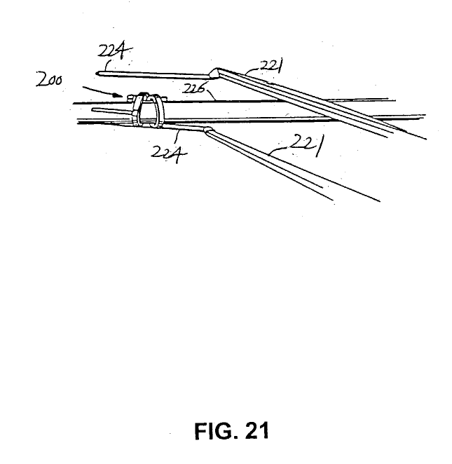

Referring to FIGS. 20-21, a peripheral nerve 226 is surgically exposed in preparation for installation of electrode assembly 200. Pins 224 of the installation tool are compressed together by squeezing the tweezer, and the adjacent pins are slipped through the hollow interior of the helical electrode assembly.

Gripping force on the tweezer is then relaxed, permitting the pins to separate and thereby open the electrode assembly so that it can be lowered over nerve 226 as shown in FIG. 20.

Tool 220 is initially positioned within the electrode assembly such that, when the pins are separated, one pin will be close to bridge portion 202, and the other pin will be adjacent to the free ends of helical portions 203 and 204. The resulting unwrapping or unwinding of flexible matrix 201 and the associated electrode or electrodes opens the helical turns to form a laterally open passage 227 to receive the nerve as shown in FIG. 22.

When the spread electrode assembly has been placed over the top of the nerve, the tweezer pins can be moved toward each other beneath the nerve, and continued lowering of the tweezer tips withdraws the pins from within the electrode. The tweezer is then sufficiently reopened to provide clearance between the pins and the nerve so the tool can be withdrawn.

When the tool pins are removed, the shape memory of resilient matrix 201 causes an automatic self-closing action of helical portions 203 and 204 around nerve 226. The preferred slight compressive fit of the helical portions places the electrode or electrodes in the desired intimate contact with the nerve for good electrical conduction of stimulating signals.

In some implantations of the electrode assembly, there may be only a very slight clearance between the under surface of the nerve and the underlying body structure. In this situation, the electrode assembly is fitted over the nerve, as already described, and the tool pins are then compressed together and gently withdrawn from the electrode matrix by a sideways movement parallel to the nerve axis. The tips are then again spread sufficiently to be withdrawn over the opposed sides of the nerve.

Separation of the installation-tool pins within the helical portions causes unwinding of the helical turns by a sliding movement of the matrix inner surface on the pins. Preferably, the pins are coated with a lubricating plastic, such as Teflon, coated to minimize frictional resistance to the sliding motion of the silicone-rubber matrix over the pins.

This embodiment of an electrode assembly according to the present invention has significant advantages of minimum interference with desirable fluid exchange between the nerve and surrounding tissue, and minimum risk of excessive nerve compression which can cause nerve damage. The assembly according to the present invention is particularly capable of resiliently accommodating nerve swelling or edema resulting from the implantation surgery. Similarly, the assembly has good longitudinal flexibility to accommodate bending of the associated nerve during limb articulation or other body movement. Good electrical contact of the electrode and nerve is also achieved, with little risk of tissue-ingrowth problems encountered with cuff electrodes.

The oppositely directed turns of the helical portions provide an important advantage of good assembly anchorage and resistance to axial movement of the assembly along the nerve in response to adjacent muscle movement or limb articulation. The anchoring effect arises from an opening separation of the distal helical portion, which reduces the matrix inside diameter to increase the gripping action of the matrix around the nerve.

The tight pitch of the helical portions, and the capability of using multiple electrodes, enable the use of multiple stimulus sites along and around the nerve for selective stimulation of nerve bundles, particularly according to the scheme described above involving selection of the helical pitch and the number of helical turns of each electrode.

Another advantage of this embodiment is ease of installation, and freedom from any need to wind the assembly manually around the nerve. In addition to reducing surgical manipulation and possible nerve trauma, the simple open-lower-close installation sequence permits placement even where the exposed nerve is deeply recessed in the body with very small undersurface clearance.

Although described above in terms of an assembly with two helical portions of opposite rotation direction, this embodiment of the invention further extends to a single helical portion that is useful where axial exposure of the nerve is limited. In both configurations, the helical turn extends around at least 360° to provide complete encirclement of the nerve, and preferably about one-half turn beyond a full nerve. If desired, the electrode ribbon can extend along the entire inner circumference of the helical matrix to provide constant stiffness, and any unwanted conductive contact is avoided by applying an insulating coating (Epoxylite is suitable) to portions of the exposed electrode surface.

The extent of the matrix helical turn is preferably kept less than two full turns for several reasons. First, the greater circumferential extent of the helical portion requires a greater separation of the insulation-tool pins to open the assembly for fitting over the nerve, and this separation should be minimized so that the tool pins can be fitted within a narrow incision. A second factor is to limit distortion of the electrode ribbon, which can decrease the desired intimate contact of the electrode against the nerve surface.

Another embodiment of the invention is a device for treating a particular condition by electrical stimulation of a particular nerve. The condition and treatment can be one of the following:

(1) treatment of obstructive sleep apnea by stimulation of the glossopharyngeal nerve;

(2) treatment of epilepsy by stimulation of the vagus nerve;

(3) producing diaphragm contractions by electrical stimulation of the phrenic nerve;

(4) restoring hand function by stimulation of a peripheral nerve in the upper extremities; and

(5) regulating gait by electrical stimulation of a peripheral nerve in the lower extremities.

In general, such a device comprises:

(1) a helical electrode assembly comprising:

(a) a flexible helically formed supporting matrix of dielectric material;

(b) at least one flexible conductive electrode secured to the surface of the matrix, the electrode having front and rear surfaces and side edges, the front surface being exposed and not covered by the matrix, the electrode occupying only a portion of the cross-sectional periphery of the matrix; and

(c) at least one flexible connector connected to the at least one electrode and extending from the matrix; the matrix and at least one electrode generally forming a multi-turn hollow helix of a selected pitch and with a selected number of helical turns of each electrode, the helix having a free end and without a supporting core, the turns of the helix being resiliently movable with respect to each other to enable the helix to be wrapped around the unsevered nerve to be treated, the helix having a central passage therethrough of size configuration generally conforming to the external size configuration of the nerve to be treated, the pitch of the helix and the number of helical turns being selected such that the entire nerve to be treated or any portion of it extending radially inward from the surface forming an annulus can be electrically stimulated to activate substantially all axons or a subpopulation of axons of the nerve;

(2) a controllable current or voltage source in electrical contact with the at least one flexible connector; and

(3) a controller for controlling the current or voltage source so that an electric field is created by the at least one electrode to treat the specified condition.

Typically, the supporting matrix is molded silicone.

Typically, as described above, the electrode is a ribbon of activated iridium.

Typically, the first surface of the electrode is roughened to increase the effective area of the surface. Preferably, the effective surface area is increased by a factor of at least about 10 as compared with a perfectly smooth surface. More preferably, the effective surface area is increased by a factor of at least about 20 as compared with a perfectly smooth surface.

The device can comprise at least one electrode or a plurality of electrodes. When the device comprises one electrode, the electrode can have a single helical turn or more than a single helical turn. When the device has a plurality of electrodes, each of the plurality of electrodes can have either a single helical turn or more than a single helical turn.

When the device comprises one electrode that has more than one helical turn, the device can further comprise a switch for selectively directing voltage to each helical turn of the electrode.

Similarly, when the device comprises a plurality of electrodes, each with more than a single helical turn, the device can further comprise a switch for selectively directing voltage to each helical turn of each electrode. Such switching mechanisms are well known in the art.

The invention is illustrated by the following example. This example is for illustrative purposes only, and is not intended to limit the invention.

### EXAMPLE

### Modeling of Relative Threshold for Activation of Nerve Fibers as Function of Helical Pitch and Number of Helical Turns

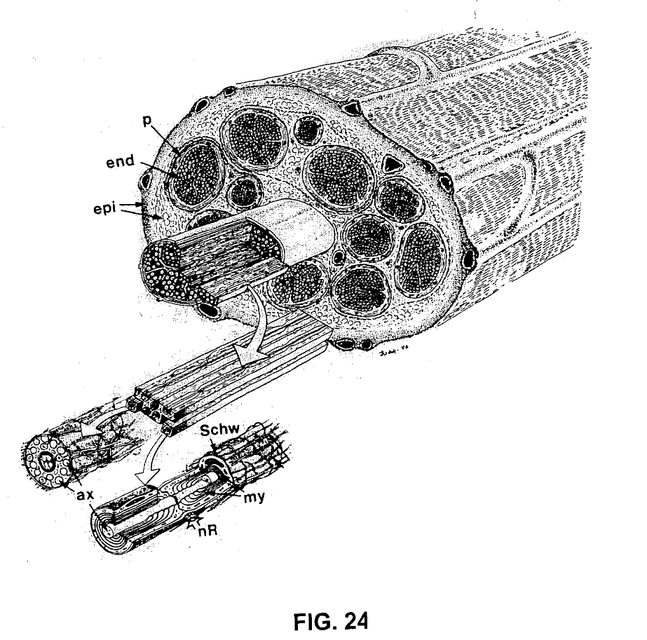

Electrical stimulation excites (activates) nerve fibers by depolarizing them. The adequate stimulus for depolarizing the fibers is the gradient of the electrical field in the direction of the fibers, the “longitudinal component of the electric field gradient”. Since nerve fibers are oriented to the axis of the nerve, as shown in FIG. 24, the adequate stimulus is the gradient of the electric field in the direction of the axis of the nerve. This is proportional to the longitudinal gradient of the current density. FIG. 24 is a diagram of the structure of a typical peripheral nerve, in which “p” is the perineurium, “end” is the endoneurium, “epi” is the epineurium, “ax” is an axon, “nR” is a node of Ranvier, “my” is myelin, and “Schw” is a Schwann cell.

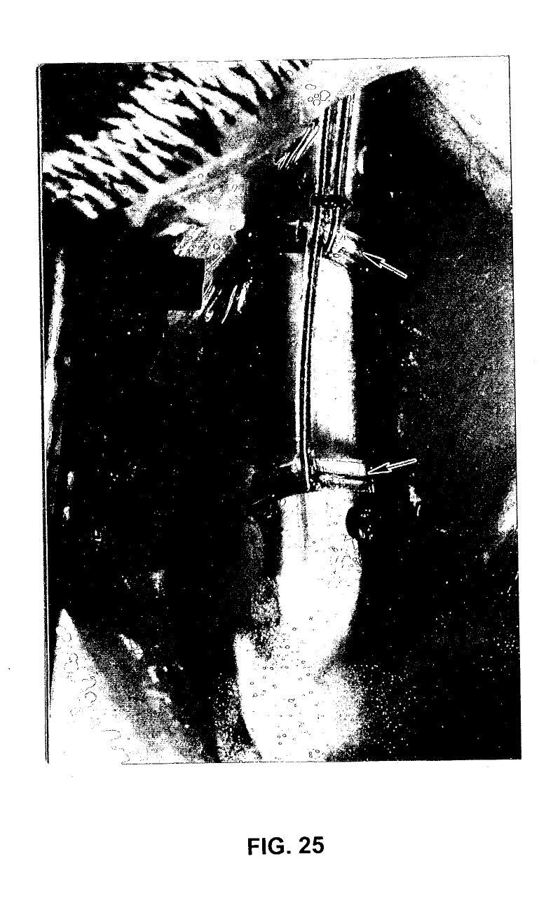

FIG. 25 is a photograph of an assembly according to the present invention installed on a peripheral nerve.

The longitudinal component of the current density induced in the nerve was modeled using a simple computer program with a finite-element protocol, in which the helical electrode band or bands were modeled as finite site-elements whose contribution was summated to yield an estimate of the total electrical field within the nerve. In the model, the nerve has radius R and one in the monopolar version, or two in the bipolar version, helical bands surround the nerve. The helix was modeled as a band of width R/4 which inscribes one complete circumference of the nerve, with the “pitch” of the helix as a parameter. A pitch of 0 is simply a circumneural ring. A helix with a pitch of 2R advances by 2R as it encircles the nerve, and so forth.

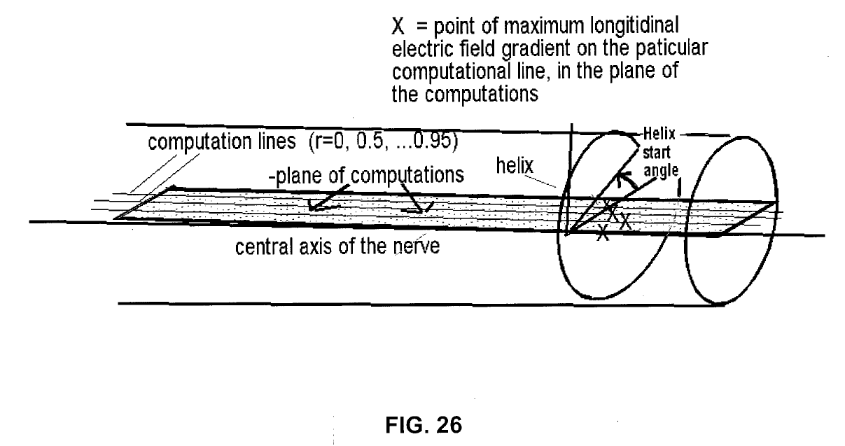

Values of the current density were computed at 800 points with the nerve, at each of 40 points along a longitudinal axis of the nerve (x-axis) for x=0 to x=4R from the start of the helix. This is shown in FIG. 26. This is repeated at each of 20 increments of radial distance from the center of the nerve. For each radial distance from the center, the maximum value of the gradient was located at some particular value of x. The action potential of the nerve will be initiated at this point at which the longitudinal gradient is maximum.

The maximum was then plotted against the radial distance from the center of the nerve. The pitch of the helix was the parameter across the family of plots, and all the plots were normalized on the maximum gradient at the center of the nerve. The reciprocal of the normalized maximum gradient also was plotted. This is an index of the relative threshold of the fibers at a particular distance from the center of the nerve. This process was repeated with the radius along which the x value is taken, but set at different angles with respect to the start of the helix. The results were found to be essentially independent of this angle of inclination.

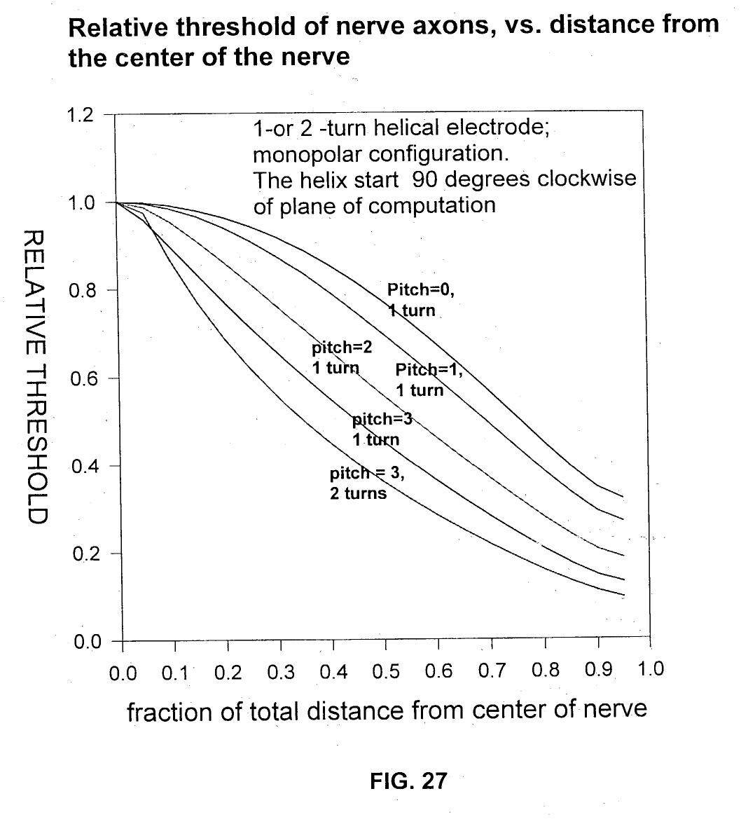

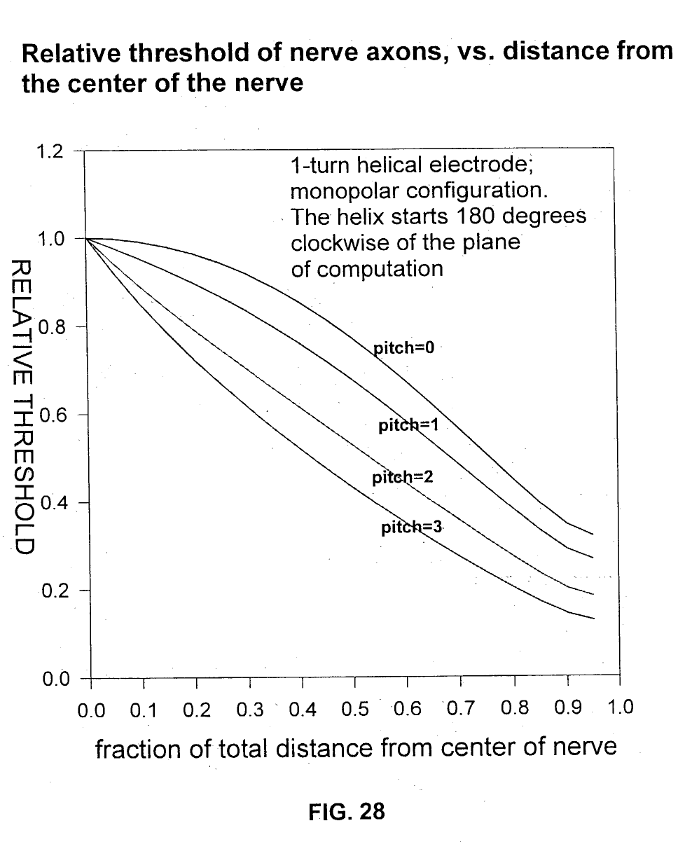

The results are shown in FIG. 27 for a one-turn helical electrode with a monopolar configuration with the helix starting 90° clockwise of the plane of computation for pitches from 0 to 3, including an example with the pitch equal to 3 and two full helical turns, and FIG. 28, for a one-turn helical electrode with a monopolar configuration where the helix starts at 180° clockwise of the plane of computation for pitches of 0 to 3. In FIGS. 27 and 28, the percent of distance from the center of the nerve is plotted along the abscissa (x-axis) while the relative threshold is plotted along the ordinate (y-axis). The model shows that as the pitch of the helix increases, there is a greater difference between the field gradient at the center and at the periphery of the nerve. Thus, if the objective is to excite all of the fibers within the nerve, the helix should have low pitch (about 1R). However, if it is desirable to be able to control a percentage of the fibers that are excited, as is desirable in a number of the applications described above, such as vagus nerve stimulation for suppression of epilepsy, then the pitch of the helix should be large (e.g., 3R). Thus, the helical electrode can be customized for either mode of operation simply by changing the pitch of the helix. The results were essentially identical for the monopolar and bipolar configurations.

What is claimed is:

1. An electrode assembly for surgical implantation on a nerve of the central or peripheral nervous system, comprising:
(a) a flexible helically formed supporting matrix of dielectric material;
(b) a flexible conductive electrode secured to the surface of the matrix, the electrode having front and rear surfaces and side edges, the front surface being exposed and not covered by the matrix, the electrode occupying only a portion of the cross-sectional periphery of the matrix; and
(c) at least one flexible connector connected to the electrode and extending from the matrix; the matrix and the electrode generally forming a multi-turn hollow helix of a selected pitch, the helix having a free end and without a supporting core, the turns of the helix being resiliently movable with respect to each other to enable the helix to be wrapped around an unsevered nerve, the helix having a central passage therethrough of size and configuration generally conforming to the external size and configuration of the nerve, the pitch being selected such that the entire nerve or any portion of it extending radially inward from its surface forming an annulus can be electrically stimulated to activate substantially all axons or a subpopulation of axons of the nerve, the size of the subpopulation being determined by the amplitude of the stimulus current.

2. The electrode assembly of claim 1 wherein the electrode is partially embedded in the matrix.

3. The electrode assembly of claim 2 wherein the matrix extends over the rear surface and side edges of the electrode.

4. The electrode assembly of claim 2 wherein one end of the electrode is folded rearwardly and the folded end is fully embedded in the matrix.

5. The electrode assembly of claim 2 wherein the flexible connector is embedded in the matrix between the electrode and an end of the matrix.

6. The electrode assembly of claim 1 wherein the front surface of the electrode is roughened to increase the effective area of the surface.

7. The electrode assembly of claim 6 wherein the degree of surface roughening of the electrode increases the effective surface area of the electrode by a factor of at least about 10 as compared with a perfectly smooth surface.

8. The electrode assembly of claim 7 wherein the degree of surface roughening of the electrode increases the effective surface area of the electrode by a factor of at least about 20 as compared with a perfectly smooth surface.

9. The electrode assembly of claim 1 wherein the flexible connector includes a flexible stranded wire.

10. The electrode assembly of claim 9 wherein the flexible connector further includes a conductive ribbon wrapped around the end of the flexible stranded wire and welded thereto to form a flattened connection tab for attachment to the electrode.

11. The electrode assembly of claim 1 wherein the matrix is molded silicone, the electrode is partially embedded in the matrix with only the electrode front surface exposed, the front surface being roughened to increase its effective area by a factor of at least about 10 as compared with a perfectly smooth surface, and the flexible connector includes a flexible stranded wire embedded in the matrix between the electrode and an end of the matrix.

12. The electrode assembly of claim 11 wherein the front surface is roughened to increase its effective area by a factor of at least about 20 as compared with a perfectly smooth surface.

13. The electrode assembly of claim 1 wherein the electrode is made of a ribbon of activated iridium, and the electrode is secured to the inner surface of a matrix to face the central axis of the matrix helix.

14. The electrode assembly of claim 1 wherein the pitch is selected such that substantially the entire nerve is stimulated and such that substantially all axons are activated at nearly the same threshold.

15. The electrode assembly of claim 1 wherein the pitch is selected such that a portion of the nerve extending radially inward from its surface forming an annulus is stimulated and such that a subpopulation of axons is activated, the size of the subpopulation being determined by the amplitude of the stimulus current.

16. The electrode assembly of claim 15 wherein the assembly is configured to stimulate the glossopharyngeal nerve to extend the tongue to treat obstructive sleep apnea.

17. The electrode assembly of claim 15 wherein the assembly is configured to stimulate the vagus nerve to treat epilepsy.

18. An electrode assembly for surgical implantation around a nerve comprising:
(a) a flexible supporting matrix formed substantially in the shape of a spiral helix; and
(b) a plurality of spaced-apart flexible conductive ribbon electrodes secured to an array along an inner surface of the matrix, each electrode occupying only a portion of the cross-sectional periphery of the matrix and having a separate flexible conductor extending therefrom; the conductors being embedded in the matrix to extend from the respective ribbon electrodes to an end of the matrix; the matrix and electrodes generally forming a multi-turn hollow helix of a selected pitch with a free end and without a supporting core, the turns of the helix being resiliently movable with respect to each other to enable the helix to be wrapped around an unsevered nerve, the helix having a central passage therethrough of size and configuration generally conforming to the external size and configuration of the nerve, the pitch being selected such that substantially the entire nerve or any portion of it extending radially inward from its surface forming an annulus can be electrically stimulated to activate substantially all axons or a subpopulation of axons.

19. The electrode assembly of claim 18 wherein the ribbon electrodes are partially embedded in the inner matrix surface with each electrode having an exposed non-embedded front surface facing a central axis of the helix, and wherein the conductors external to the matrix form a flexible connecting cable.

20. The electrode assembly of claim 19 wherein the front surfaces of the electrodes are roughened to provide an increased effective surface area.

21. The electrode assembly of claim 20 wherein the effective surface area is increased by a factor of at least about 10 as compared with a perfectly smooth surface.

22. The electrode assembly of claim 21 wherein the effective surface area is increased by a factor of at least about 20 as compared with a perfectly smooth surface.

23. The electrode assembly of claim 18 wherein the assembly further comprises an implantable biomedical electronic signal device connected to the cable of the assembly.

24. The electrode assembly of claim 18 wherein the electrodes are made of ribbons of activated iridium.

25. The electrode assembly of claim 18 wherein the pitch of the helices is selected such that substantially the entire nerve is stimulated and such that substantially all axons are activated.

26. The electrode assembly of claim 18 wherein the pitch of the helices is selected such that a portion of the nerve extending radially inward from its surface forming an annulus is stimulated and such that a subpopulation of axons is activated.

27. The electrode assembly of claim 26 wherein the assembly is configured to stimulate the glossopharyngeal nerve to extend the tongue to treat obstructive sleep apnea.

28. The electrode assembly of claim 26 wherein the assembly is configured to stimulate the vagus nerve to treat epilepsy.

29. An electrode assembly for surgical implantation on a nerve of the peripheral or central nervous system comprising:
(a) a flexible helically formed supporting matrix of dielectric material;
(b) a flexible conductive electrode secured to the surface of the matrix, the electrode having front and rear surfaces and side edges, the front surface being exposed and not covered by the matrix, the electrode occupying only a portion of the cross-sectional periphery of the matrix;
(c) at least one flexible connector connected to the electrode and extending from the matrix; the matrix and electrode generally forming a multi-turn hollow helix of a selected pitch and with a selected number of helical turns of the electrode, the helix having a free end and without a supporting core, the turns of the helix being resiliently movable with respect to each other to enable the helix to be wrapped around an unsevered nerve, the helix having a central passage therethrough of size and configuration generally conforming to the external size and configuration of the nerve, the pitch of the helix and the number of helical turns being selected such that the entire nerve or any portion of it extending radially inward from the surface forming an annulus can be electrically stimulated to activate substantially all axons or a subpopulation of axons.

30. The electrode assembly of claim 29 wherein the electrode is partially embedded in the matrix.

31. The electrode assembly of claim 30 wherein the matrix extends over the rear surface and side edges of the electrode.

32. The electrode assembly of claim 30 wherein one end of the electrode is folded rearwardly and the folded end is fully embedded in the matrix.

33. The electrode assembly of claim 30 wherein the at least one flexible connector is embedded in the matrix between the electrode and an end of the matrix.

34. The electrode assembly of claim 29 wherein the front surface of the electrode is roughened to increase the effective area of the surface.

35. The electrode assembly of claim 34 wherein the degree of surface roughening increases the effective surface area by a factor of at least about 10 as compared with a perfectly smooth surface.

36. The electrode assembly of claim 35 wherein the degree of surface roughening increases the effective surface area by a factor of at least about 20 as compared with a perfectly smooth surface.

37. The electrode assembly of claim 29 wherein the at least one flexible connector includes a flexible stranded wire.

38. The electrode assembly of claim 37 wherein the at least one flexible connector further includes a conductive ribbon wrapped around an end of the wire and welded thereto to form a flattened connection tab for attachment to the electrode.

39. The electrode assembly of claim 29 wherein the matrix is molded silicone, the electrode is partially embedded in the matrix with only the electrode front surface exposed, the front surface being roughened to increase its effective area by a factor of at least about 20 is compared with a perfectly smooth surface, and the at least one flexible connector includes a flexible stranded wire embedded in the matrix between the electrode and an end of the matrix.

40. The electrode assembly of claim 29 wherein the electrode is made of a ribbon of activated iridium, and the electrode is secured to the inner surface of the matrix to face the central axis of the matrix helix.

41. The electrode assembly of claim 29 wherein the pitch of the helix and the number of helical turns of the electrode are selected such that substantially the entire nerve is stimulated and such that substantially all axons are activated.

42. The electrode assembly of claim 29 wherein the pitch of the helix and the number of helical turns of the electrode are selected such that a portion of the nerve extending radially inward from the surface forming an annulus is electrically stimulated to activate a subpopulation of axons.

43. The electrode assembly of claim 42 wherein the assembly is configured to stimulate the glossopharyngeal nerve to extend the tongue to treat obstructive sleep apnea.

44. The electrode assembly of claim 42 wherein the assembly is configured to stimulate the vagus nerve to treat epilepsy.

45. The electrode assembly of claim 29 wherein each turn of the plurality of helical turns is electrically isolated and connected to a separate connector and wherein the assembly further comprises a switch for selectively applying voltage to one or more of the helical turns as controlled by the switch.

46. An electrode assembly for surgical implantation on a nerve of the peripheral nervous system comprising:
(a) a flexible supporting matrix formed substantially in the shape of a spiral helix; and
(b) a plurality of spaced-apart flexible conductive ribbon electrodes secured to an arranged along an inner surface of the matrix, each electrode occupying only a portion of the cross-sectional periphery of the matrix and having a separate flexible conductor extending therefrom; the conductors being embedded in the matrix to extend from the respective ribbon electrodes to an end of the matrix; the matrix and electrode generally forming a multi-turn hollow helix of a selected pitch and with a selected number of helical turns of each electrode, the helix having a free end and without a supporting core, the turns of the helix being resiliently movable with respect to each other to enable the helix to be wrapped around an unsevered nerve, the helix having a central passage therethrough of size configuration generally conforming to the external size and configuration of the nerve, the pitch of the helix and the number of helical turns of each electrode being selected such that substantially the entire nerve or any portion of it extending radially inward from the surface forming an annulus can be selectively stimulated to activate substantially all axons or a subpopulation of axons.

47. The electrode assembly of claim 46 wherein the ribbon electrodes are partially embedded in the inner matrix surface with each electrode having an exposed non-embedded front surface facing a central axis of the helix, and wherein the conductors external to the matrix form a flexible connecting cable.

48. The electrode assembly of claim 46 wherein the front surfaces of the electrodes are roughened to provide an increased effective surface area.

49. The electrode assembly of claim 48 wherein the effective surface area is increased by a factor of at least about 10 as compared with a perfectly smooth surface.

50. The electrode assembly of claim 49 wherein the effective surface area is increased by a factor of at least about 20 is compared with a perfectly smooth surface.

51. The electrode assembly of claim 46 wherein the assembly further comprises an implantable biomedical electronic signal device connected to the cable of the assembly.

52. The electrode assembly of claim 46 wherein the electrodes are made of ribbons of activated iridium.

53. The electrode assembly of claim 46 wherein the pitch of the helix and the number of helical turns of each electrode are selected such that substantially the entire nerve is stimulated and such that substantially all axons are activated.

54. The electrode assembly of claim 46 wherein the pitch of the helix and the number of helical turns of each electrode is selected such that a portion of the nerve extending radially inward from its surface forming an annulus is electrically stimulated to activate a subpopulation of axons.

55. The electrode assembly of claim 54 wherein the assembly is configured to stimulate the glossopharyngeal nerve to extend the tongue to treat obstructive sleep apnea.

56. The electrode assembly of claim 54 wherein the assembly is configured to stimulate the vagus nerve to treat epilepsy.

57. The electrode assembly of claim 46 wherein each turn of the plurality of helical turns of each electrode is electrically isolated and connected to a separate connector and wherein the assembly further comprises a switch for selectively applying voltage to one or more of the helical turns of each electrode as controlled by the switch.

58. A device for treating a condition or disease treatable by electrical stimulation of a peripheral nerve comprising:
(a) a helical electrode assembly comprising:
(i) a flexible helically formed supporting matrix of dielectric material;
(ii) at least one flexible conductive electrode secured to the surface of the matrix, the electrode having front and rear surfaces and side edges, the front surface being exposed and not covered by the matrix, the electrode occupying only a portion of the cross-sectional periphery of the matrix; and
(iii) at least one flexible connector connected to the at least one electrode and extending from the matrix; the matrix and the at least one electrode generally forming a multi-turn hollow helix of a selected pitch and with a selected number of helical turns of each electrode, the helix having a free end and without a supporting core, the turns of the helix being resiliently movable with respect to each other to enable the helix to be wrapped around an unsevered peripheral nerve whose stimulation treats the disease or condition, the helix having a central passage therethrough of size and configuration generally conforming to the external size and configuration of the peripheral nerve, the pitch of the helix and the number of helical turns being selected such that the entire peripheral nerve or any portion of it extending radially inward from the surface forming an annulus can be electrically stimulated to activate substantially all axons or a subpopulation of axons of the nerve;
(b) a controllable current or voltage source in electrical contact with the at least one flexible connector; and
(c) a controller for controlling the current or voltage source so that an electric field is created by the at least one electrode to treat the disease or condition.

59. The device of claim 58 wherein the supporting matrix is molded silicone.

60. The device of claim 58 wherein the electrode is a ribbon of activated iridium.

61. The device of claim 58 wherein the first surface of the electrode is roughened to increase the effective area of the surface.

62. The device of claim 61 wherein the effective surface area is increased by a factor of at least about 10 as compared with a perfectly smooth surface.

63. The device of claim 58 wherein the device comprises one electrode.

64. The device of claim 58 wherein the device comprises a plurality of electrodes.

65. The device of claim 63 wherein the electrode has a single helical turn.

66. The device of claim 63 wherein the electrode has more than a single helical turn.

67. The device of claim 64 wherein each of the plurality of electrodes has a single helical turn.

68. The device of claim 64 wherein each of the plurality of electrodes has more than a single helical turn.

69. The device of claim 66 further comprising a switch for selectively directing voltage to each helical turn of the electrode.

70. The device of claim 68 further comprising a switch for selectively directing voltage to each helical turn of each electrode.

71. The device of claim 58 wherein the nerve to be simulated is selected from the group consisting of: (1) the glossopharyngeal nerve to treat obstructive sleep apnea; (2) the vagus nerve to treat epilepsy; (3) the auditory nerve to restore hearing; (4) the phrenic nerve to produce diaphragm convulsions; (5) a peripheral nerve in the upper extremities to restore hand function; and (6) a peripheral nerve in the upper extremities to regulate gait.

72. The device of claim 71 wherein the nerve to be stimulated is the glossopharyngeal nerve to treat obstructive sleep apnea.

73. The device of claim 71 wherein the nerve to be stimulated is the vagus nerve to treat epilepsy.

74. The device of claim 71 wherein the nerve to be stimulated is the auditory nerve to restore hearing.

75. The device of claim 71 wherein the nerve to be stimulated is the phrenic nerve to produce diaphragm convulsions.

76. The device of claim 71 wherein the nerve to be stimulated is a peripheral nerve in the upper extremities to restore hand function.

77. The device of claim 71 wherein the nerve to be stimulated is a peripheral nerve in the lower extremities to regulate gait.

78. An electrode assembly for implantation on a nerve comprising:
(a) a flexible supporting matrix of dielectric material, the matrix forming a pair of spaced-apart and oppositely directed helical portions, each helical portion extending circumferentially at least 360° and less than 720°;
(b) a flexible conductive electrode secured to an inner surface of one of the helical portions, the flexible conductible electrode forming a helix of selected pitch; and
(c) a flexible connector connected to the electrode and extending from the matrix for connection to an electronic device; the assembly having a central passage longitudinally through and sized to conform to the external dimension of the nerve, whereby a tool can be inserted in the passage to expand the helical portions to open a lateral passage along the full length of the assembly to enable the assembly to be fitted over and closed upon the nerve; the pitch of the helix being selected such that the entire nerve or any portion of it extending radially inward from its surface forming an annulus can be electrically stimulated to activate substantially all axons or a subpopulation of axons.

79. The electrode assembly of claim 78 wherein the helical portions each have adjacent turns that are spaced apart less than the axial width of the matrix to minimize the axial length of the assembly while providing space between the adjacent turns to permit fluid passage to the nerve.

80. The electrode assembly of claim 78 wherein the helical portions are joined by a matrix bridge portion which extends generally parallel to a central axis of the helical portions.

81. The electrode assembly of claim 78 wherein a flexible conductive electrode is secured to the inner surface of each helical portion, and the flexible connector comprises stranded wires secured to the respective electrodes and extending from the outer surface of the respective helical portions.

82. The electrode assembly of claim 78 wherein each helical portion extends circumferentially about 1.5 turns.

83. The electrode assembly of claim 78 wherein each helical portion extends circumferentially in the range of from about 420 to about 540 degrees.

84. The electrode assembly of claim 78 wherein the electrode is activated iridium.

85. The electrode assembly of claim 78 wherein the matrix is molded silicone.

86. A kit comprising:
(a) an electrode assembly for implantation on a nerve comprising:
(i) a flexible supporting matrix of dielectric material, the matrix forming a pair of spaced-apart and oppositely directed helical portions, each helical portion extending circumferentially at least 360° and less than 720°;
(ii) a flexible conductive electrode secured to an inner surface of one of the helical portions, a flexible conductive electrode forming a helix of defined pitch; and
(iii) a flexible connector connected to the electrode and extending from the matrix for connection to an electronic device; the pitch of the helix being selected such that the entire nerve or any portion of it extending radially inward from its surface forming an annulus can be electrically stimulated to activate substantially all axons or a subpopulation of axons; and
(b) an insertion tool having a portion which is fitted and expanded within the central passage to expand the helical portion and thereby to form a laterally open passage along the length of the assembly so that the assembly can be fitted over and closed upon the nerve upon removal of the tool portion.

87. The kit of claim 86 wherein the insertion tool has a pair of separable legs, each leg having a free and defining pin, the pins being generally parallel and juxtaposed when the legs are moved toward each other so that the pins can be inserted and expanded within the electrode assembly.

88. The kit of claim 87 wherein the tool pins are oriented at an angle to longitudinal axes of the respective legs.

89. The kit of claim 88 wherein the angle is about 45°.

90. The kit of claim 87 wherein each pin defines a concave depression for receiving the matrix.
